(19) United States
(12) Patent Application Publication
(10) Pub. No.: US 2025/0242030 A1
(43) Pub. Date: Jul. 31, 2025

(54) COMPOSITIONS COMPRISING A NON-BIOABSORBABLE POLYMER AND METABOLIC INHIBITOR

(71) Applicants: Board of Trustees of Michigan State University, East Lansing, MI (US); THE BOARD OF TRUSTEES OF THE LELAND STANFORD JUNIOR UNIVERSITY, Stanford, CA (US)

(72) Inventors: Chima V. MADUKA, Westminster, CO (US); Christopher CONTAG, East Lansing, MI (US); Stuart GOODMAN, Los Altos, CA (US)

(73) Assignees: Board of Trustees of Michigan State University, East Lansing, MI (US); THE BOARD OF TRUSTEES OF THE LELAND STANFORD JUNIOR UNIVERSITY, Stanford, CA (US)

(21) Appl. No.: 18/833,739

(22) PCT Filed: Jan. 27, 2023
(86) PCT No.: PCT/US2023/011740

§ 371 Date: Jul. 26, 2024

Related U.S. Application Data
(60) Provisional application No. 63/303,757, filed on Jan. 27, 2022.

(51) Int. Cl. A61K 47/32; A61K 31/155; A61K 31/195; A61K 31/35; A61K 31/444; A61K 31/7004; A61K 47/58

(52) U.S. Cl. CPC A61K 47/32; A61K 31/155; A61K 31/195; A61K 31/35; A61K 31/444; A61K 31/7004; A61K 47/58

(57) ABSTRACT

Disclosed herein are compositions comprising a polymer and a metabolic inhibitor, as well as a method of using the composition to modulate an immune response. The composition may be produced in the form of a synthetic tissue for provision in a subject. The composition or synthetic tissue may further comprise additional therapeutic agents.

### CROSS-REFERENCE TO RELATED APPLICATIONS

This application claims the benefit under 35 U.S.C. § 119(e) to U.S. Provisional Application No. 63/303,757 filed on 27 Jan. 2022. The entire contents of U.S. 63/303,757 are hereby incorporated by reference in their entirety.

### GOVERNMENTAL RIGHTS

This invention was made with government support under AR073145 and AR063717, awarded by the NIH. The government has certain rights in the invention

### FIELD

The field of the invention relates to compositions comprising a polymer and a metabolic inhibitor. The composition may be used as a synthetic tissue or prosthetic that may modulate an immune response.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

End-stage arthritis can be successfully treated by primary total joint arthroplasties (TJAs). With nearly 50% of TJAs performed in patient younger than 65 years, the vision of TJAs is not to reconstruct joints which will last a lifetime, despite patient's daily activities. This is useful because revision TJAs are costly and fraught with higher complication rates, technical difficulties, and poor surgical outcomes as compared to primary TJAs. Revision TJAs commonly arise from aseptic loosening, frequently incited by polyethylene wear particles generated by relative motion at joint articulations. Aseptic loosening may occur with or without adsorbed contaminants, such as bacterial lipopolysaccharides (LPS). Wear particles induce prolonged, low-grade inflammation with macrophages and fibroblasts as key immune cellular players. This pathology is often radiographically detected only when surrounding bone loss (periprosthetic osteolysis) occurs. By then, compromised implant stability results in loosening and implant failure, thereby necessitating revision surgeries.

To minimize generation of wear particles, ultrahigh molecular weight polyethylene liners at the bearing surfaces of reconstructed joints are currently being replaced by highly crosslinked polyethylene. Crosslinked polyethylene has significantly reduced the amount of generated wear particles and accompanied chronic inflammation with periprosthetic osteolysis. However, crosslinking does not completely block the generation of wear particles from bearing surfaces of implants and subsequent inflammation. Up to 9% of patients with crosslinked polyethylene liners present with chronic inflammation-induced periprosthetic osteolysis 15 years later. Moreover, crosslinking has little effect on particles from third body wear, backside wear and impingement;

and there are currently no agents that specifically treat polyethylene particle-induced inflammatory osteolysis. Consequently, there is an unmet clinical need to develop methods that will mitigate aseptic loosening from polyethylene particle-induced chronic inflammation to improve implant longevity. Furthermore, as particles generated from ultrahigh molecular weight or highly crosslinked polyethylene similarly result in inflammation, either of the effectively models particle-induced inflammation.

Metabolic reprogramming refers to changes in glycolytic flux and oxidative phosphorylation (OXPHOS), traditional bioenergetic pathways, that are inextricably linked to macrophage activation toward proinflammatory or pro-regenerative phenotypes. Advances in understanding macrophage-mesenchymal stem cell crosstalk has revealed that down modulating inflammation induced by polyethylene particles can prevent implant loosening by enhancing osscointegration through increased pro-regenerative macrophage activity. For example, using mesenchymal stem cells (MSCs) and engineered IL-4 expressing MSCs18; targeting inflammatory pathways using decoy molecules for NF-kB, TNF-α and MCP-1; and using antioxidants like vitamin E have shown promise for enhanced osscointegration by reducing inflammation. However, the metabolic underpinnings underlying macrophage activation by polyethylene particles are largely undefined. A detailed understanding of metabolic programs could be leveraged for immunomodulation toward extending the longevity of implants. Here, we show that both macrophages and fibroblasts exposed to sterile or LPS-contaminated polyethylene particles undergo metabolic reprogramming and differential changes in bioenergetics. Changes in glycolytic flux and mitochondrial respiration in the electron transport chain (ETC) underlie the increased levels of proinflammatory cytokines, including MCP-1, IL-6, IL-1β and TNF-α. Specific inhibition of different glycolytic steps not only modulated these proinflammatory cytokines but stimulated pro-regenerative cytokines, including IL-4 and IL-10, without affecting cell viability. Similar immunomodulatory effects were obtained by targeting complex I of the mitochondrial ETC. Concomitant elevation of both glycolytic flux and oxidative phosphorylation by polyethylene particles and inhibitory effects on inflammatory cytokines, in addition to IL-1β,suggest a unique metabolic program that could be targeted for pro-regenerative clinical outcomes following TJAs.

### SUMMARY

A composition comprising a polymer and a metabolic inhibitor is disclosed herein. The polymer may be a non-bioabsorbable polymer and/or a synthetic polymer, such as a polyethylene, a polyethylene copolymer, or a mixture of two or more polymers. The metabolic inhibitor may be a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, an inhibitor of the mitochondrial electronic transport chain (mETC inhibitor), or a combination of one or more of these or other metabolic inhibitors. The composition may be in the form of a synthetic tissue (e.g., a synthetic bone, synthetic cartilage, synthetic tendon, synthetic skin, synthetic blood, synthetic kidney, or synthetic liver). Alternatively, the composition may be in the form of a depot, such as a drug depot, in which metabolic inhibitor is released from the depot as the polymer breaks down. The composition may further include an additional therapeutic agent.

A method for modulating an immune response is also described herein. The method comprises providing to a subject in need thereof (e.g., a patient experiencing an undesired immune response), a composition comprising a polymer and a metabolic inhibitor. The polymer may be a non-bioabsorbable polymer or a synthetic polymer, such as polyethylene, a polyethylene copolymer, or a combination thereof. The metabolic inhibitor may be a PFKFB3 inhibitor, a glycolytic inhibitor, a biguanide, a GABA-T inhibitor, an mETC inhibitor, or a combination of one or more of the metabolic inhibitors. The composition provided to the subject may be in the form of a synthetic tissue (e.g., a synthetic bone, synthetic cartilage, synthetic tendon, synthetic skin, blood, kidney, or liver) located an any appropriate location within the body. Alternatively, the subject may receive a depot, such as a drug depot, in which a metabolic inhibitor is released from the depot as the polymer (e.g., the non-bioabsorbable polymer or synthetic polymer) breaks down. The method may further include an additional therapeutic agent. The subject may be a human or a non-human animal.

A synthetic tissue comprising a polymer and a metabolic inhibitor is also described herein. The synthetic tissue is designed to mimic an existing or removed body part in size, structure and/or function. The synthetic tissue is not limited may be in the form of a bone, cartilage, tendon, skin, blood, kidney, or liver. The polymer of the synthetic tissue may be a non-bioabsorbable polymer or a synthetic polymer, such as polyethylene, a polyethylene copolymer, or a combination thereof. The metabolic inhibitor may be a PFKFB3 inhibitor, a glycolytic inhibitor, a biguanide, a GABA-T inhibitor, an mETC inhibitor, or a combination of one or more of the metabolic inhibitors.

Depots, such as drug depots, comprising a polymer and a metabolic inhibitor are also described herein. The depots do not take on the size, structure, and/or function of a body part and are provided in a therapeutically suitable location of the body, including but not limited to the vascular system. The depot breaks down over time to release the metabolic inhibitor and, in some embodiments, an additional therapeutic agent. The polymer of the depot may be a non-bioabsorbable polymer or a synthetic polymer, such as polyethylene, a polyethylene copolymer, or a combination thereof. The metabolic inhibitor may be a PFKFB3 inhibitor, a glycolytic inhibitor, a biguanide, a GABA-T inhibitor, an mETC inhibitor, or a combinations of one or more of the metabolic inhibitors.

Various objects, features, aspects, and advantages will become more apparent from the following detailed description of preferred embodiments, along with the accompanying drawings.

### BRIEF DESCRIPTION OF THE DRAWINGS

FIG. 1 depicts total ATP level of untreated mouse embryonic fibroblasts (MEFs) or MEFs following exposure to ultra-high molecular weight polyethylene (PE) particles, endotoxin (LPS) and LPS-contaminated PE particles.

FIG. 2 depicts total ATP level of untreated bone marrow-derived macrophages (BMDMs) or BMDMs following exposure to ultra-high molecular weight polyethylene (PE) particles, endotoxin (LPS) and LPS-contaminated PE particles.

FIG. 3 depicts the oxygen consumption rate (OCR), extracellular acidification rate (ECAR) and lactate-linked proton efflux rate (PER) of MEFs following exposure to ultra-high molecular weight polyethylene (PE) particles, endotoxin (LPS) and LPS-contaminated PE particles.

FIG. 4 depicts the oxygen consumption rate (OCR), extracellular acidification rate (ECAR) and lactate-linked proton efflux rate (PER) of BMDMs following exposure to ultra-high molecular weight polyethylene particles (PE) in the absence and presence of metabolic inhibitors.

FIG. 5 depicts total ATP levels of MEFs treated with ultra-high molecular weight polyethylene particles (PE) in the absence and presence of metabolic inhibitors.

FIG. 6 depicts the effect of metabolic inhibitors on expression of various cytokines in BMDMs. FIG. 6a depicts expression of MCP-1. FIG. 6b depicts expression of IL-6. FIG. 6c depicts expression of IL-1β. FIG. 6d depicts expression of TNF-α. FIG. 6e depicts expression of IL-4. FIG. 6f depicts expression of IL-10.

FIG. 7 depicts ATP level of BMDMs (FIG. 7a) or MEFs (FIG. 7b) following exposure to ultra-high molecular weight polyethylene particles (PE) in the absence or presence of metabolic inhibitors.

FIG. 8 depicts the ECAR, PER, and OCR of BMDMs following exposure to ultra-high molecular weight polyethylene particles (PE) in the absence or presence of metabolic inhibitors.

FIG. 9 depicts the mean fluorescence intensities for mitoSOX and TMRM in untreated and PE-treated BMDMs.

FIG. 10 depicts the effect of metformin on the expression various cytokines in BMDMs treated with ultra-high molecular weight polyethylene particles (PE). FIG. 10a depicts expression of IL-1β. FIG. 10b depicts expression of IL-6. FIG. 10c depicts expression of MCP-1. FIG. 10d depicts expression of TNF-α. FIG. 10e depicts expression of IL-4. FIG. 10f depicts expression of IL-10.

### DETAILED DESCRIPTION

### Definitions

The following definitions refer to the various terms used above and throughout the disclosure.

As used hercin, all nouns in singular form are intended to convey the plural and all nouns in plural form are intended to convey the singular, except where context clearly indicates otherwise. As used herein, “and/or” includes any and all combinations of one or more of the associated listed items.

As used herein, a “non-bioabsorbable polymer” refers to a polymer which is not actively degraded when exposed to and/or placed within a biological environment, such as on or within the body of an animal or placed under conditions that simulate or mimic a biological environment. However, over time, the non-bioabsorbable polymer may break down, such as due to age. The non-bioabsorbable polymer maybe a synthetic polymer. The non-bioabsorbable polymer is not particularly limited, and may be a polyethylene, a polyethylene copolymer, or a combination thereof. Where the non-bioabsorbable polymer is a chiral molecule, the non-bioabsorbable polymer encompasses racemic or stereo complex mixtures of the polymer (e.g., a 50/50 mixture of both stercoisomers), or a mixture enriched for a specific stereoisomer (c.g., an 80/20 mixture of the D- and L-stereoisomers, respectively).

As used herein, “metabolic inhibitor” refers to a compound that inhibits, halts, or otherwise interferes with the ATP-producing pathways of a cell. The metabolic pathway encompasses numerous reaction schemes, such as glycolysis, glycogenolysis, fatty acid oxidation, amino acid oxidation, the Krebs cycle, the pentose phosphate pathway, and the electron transport system. A metabolic inhibitor is an agent that inhibits one or more steps of the one or more of the reaction schemes and reduces production of ATP. Metabolic inhibitors include, but are not limited to, PFKFB3 inhibitors that inhibit 6-phosphofructo-2-kinase/fructose-2,6-bisphosphatase 3, glycolytic inhibitors that inhibit glycolysis, biguanides that may enhance cellular glucose uptake to inhibit gluconcogenesis, GABA-T inhibitors that inhibit γ-aminobutyrate aminotransferase, and inhibitors of the mitochondrial electron transport chain. Specific PFKFB3 inhibitors may be 3-(3-pyridinyl)-1-(4-pyridinyl)-2-propen-1-onc (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15). Specific glycolytic inhibitors may be 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, or NHI1. Specific biguanides may be metformin, buformin, or phenoformin. Specific GABA-T inhibitors may be aminooxyacetic acid, vigabatrin, gabaculine, phenelzine, phenylethylidinehydrazine (PEH), rosmarinic acid, valproic acid, ethanolamine-O-sulfate (EOS), or cycloserine. mETC inhibitors include rotenoids and macrolides. Specific rotenoids may be rotenone, rotenol, deguelin, dehydrodegulein, tephrosin, or sumatrol. Specific macrolides include oligomycin (c.g., oligomycin A, oligomycin B, oligomycin C, oligomycin D, oligomycin E, oligomycin F, rutamycin B, 44-homooligomycin A, or 44-homooligomycin B), azithromycin, clarithromycin, or erythromycin. Other particular mETC inhibitors include trifluoromethoxy carbonylcyanide phenylhydrazone (FCCP), and related drugs.

Alternatively, the metabolic inhibitor may be something other than a small molecule inhibitor. For example, the metabolic inhibitor may be nucleic acid that inhibits expression of an enzyme involved in the metabolic pathway (c.g., mRNA. RNAi, siRNA, miRNA, dsRNA). Additionally or alternatively, the metabolic inhibitor may be a gene that encodes the production of a protein (e.g., an antibody or an antigen binding fragment) that inhibits an enzyme involved in the metabolic pathway. The gene may be a synthetic, engineered, or natural gene (e.g., DNA). In some embodiments, the metabolic inhibitor is the antibody or antigen binding fragment itself.

As used herein, “effective amount” refers to the amount, dosage, and/or dosage regime of the metabolic inhibitor in the composition, synthetic tissue, or depot that is sufficient to induce a desired clinical and/or therapeutic outcome. The effective amount may also refer to the amount, dosage, and/or dosage regime of an additional therapeutic agent.

As used herein, “immune response” represents the action of one or more components of an immune system in reaction to one or more stimuli. The immune response may occur within a body of an animal (e.g., a human), outside the body of an animal (e.g., an ex vivo tissue), or in an in vitro environment that mimics the immune response. The immune response includes both the innate and the adaptive immune systems. Modulating an immune response includes both enhancing an immune response or inhibiting an immune response. Enhancing an immune response may include increasing expression and/or release of pro-inflammatory cytokines (e.g., IL-1, TNF-α, IL-6, and IFN-γ), increasing the inflammatory activity of immune cells, decreasing expression and/or release of anti-inflammatory cytokines (e.g., IL-4, IL-13, IL-10, and TGF-β), and/or decreasing the inflammatory activity of regulatory cells. Inhibiting an immune response may include decreasing expression and/or release of pro-inflammatory cytokines, decreasing the inflammatory activity of immune cells, increasing expression and/or release of anti-inflammatory cytokines, and/or increasing the activity of regulatory cells. The immune response may be in response to the breakdown or degradation of a polymer, such as a non-bioabsorbable polymer or a synthetic polymer, such as polyethylene, a polyethylene copolymer, or a combination thereof.

As used herein, “synthetic tissue” refers to a composition that mimics the structure, shape, and/or function of an endogenous organ, tissue, cell, blood cell, body part, or part thereof. The synthetic tissue composition comprises a polymer (e.g., a non-biodegradable polymer) and a metabolic inhibitor. The synthetic tissue may mimic one or more cells, organs, tissues, body part, or part thereof, including, but not limited to, bone, cartilage, tendons, skin, blood, kidney, and liver. The synthetic tissue may replace an organ, tissue, or body part that has been removed in a subject. Alternatively, the synthetic tissue may replace or fill in a void created by the absence of a portion of an organ, tissue, and/or body part. In such instances, the synthetic tissue may be grafted onto a damaged or partially removed organ, tissue, and/or body part. Alternatively, the synthetic tissue may be inserted into an area of the body that is lacking a particular organ, tissue, and/or body part. The synthetic tissue may further comprise an additional therapeutic agent.

A “depot” is distinguished from a synthetic tissue in that the depot does not mimic or replace the structure and/or function of an organ. A depot, then, is comprised of a polymer (c.g., a non-biodegradable polymer) and a metabolic agent, wherein the metabolic agent is released as the polymer degrades or through osmotic pressure.

As used herein, “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker. The subject may have undergone surgery, received orthopedic treatment, received ophthalmic treatment, or suffering from injury or chronic disease. Alternatively, where the subject is a laboratory mammal, the composition, synthetic tissue, or depot may be provided to the laboratory mammal to achieve a scientific understanding rather than a clinical benefit.

### Compositions

The compositions described herein may comprise a polymer, such as a non-bioabsorbable polymer, or a combination of polymers, and one or more metabolic inhibitors. The non-bioabsorbable polymer may be a synthetic polymer, such as one or more of polyethylene, polyethylene copolymer, or a combination thereof. The polymer or combination of polymers may be further combined with or mixed with biologically acceptable metals and/or ceramics. The metabolic inhibitor may be a PFKFB3 inhibitor, a glycolytic inhibitor, a modulator and/or inhibitor of the mitochondrial electron transport chain (ETC), a biguanide, a GABA-T inhibitor, a rotenoid, a macrolide, or combinations thereof. The polymer may include stereoisomers of the polymer.

The composition may comprise an amount of the metabolic inhibitor as low as about 1 μM and as high as about 1 M, or any amount in between, such as, about 10 μM, about 100 μM, about 0.25 mM, about 0.5 mM, about 1 mM, about 10 mM, about 15 mM, about 20 mM, about 25 mM, about 50 mM, and about 100 mM. The composition may comprise a range of metabolic inhibitor, such as between about 1 μM and about 1 M. Any range in between about 1 μM and about 1 M is also contemplated, including, but not limited to, between about 10 μM and about 100 mM, between about 100 μM and about 20 mM, or, in particular, between about 0.5 mM and about 15 mM.

The composition may comprise a PFKFB3 inhibitor as the metabolic inhibitor. The PFKFB3 inhibitor may be one or more of 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15). In a preferred embodiment, the metabolic inhibitor is 3PO and accounts for between about 0.02 and about 21 wt % of the total weight of the composition.

The composition may comprise a glycolytic inhibitor as the metabolic inhibitor. The glycolytic inhibitor may be one or more of 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, or NHI1. In a preferred embodiment, the metabolic inhibitor is 2DG and accounts for between about 0.01 and about 17 wt % of the total weight of the composition.

The composition may comprise a biguanide as the metabolic inhibitor. The biguanide may be one or more of metformin, buformin, or phenoformin. In a preferred embodiment, the metabolic inhibitor is metformin and accounts for between about 0.01 and about 11 wt % of the total weight of the composition.

The composition may comprise a GABA-T inhibitor as the metabolic inhibitor. The GABA-T inhibitor may be one or more of aminooxyacetic acid, vigabatrin, gabaculine, phenelzine, phenylethylidinchydrazine (PEH), rosmarinic acid, valproic acid, ethanolamine-O-sulfate (EOS), and cycloserine. In a preferred embodiment, the metabolic inhibitor is metformin and accounts for between about 0.01 and about 13 wt % of the total weight of the composition.

The composition may comprise a mETC inhibitor as the metabolic inhibitor. The mETC may be a rotenoid or a macrolide as the metabolic inhibitor. The rotenoid may be one or more of rotenone, rotenol, deguelin, dehydrodegulein, tephrosin, or sumatrol. The macrolide may be one or more of oligomycin, azithromycin, clarithromycin, or erythromycin. Alternatively, the mETC inhibitor may be a trifluoromethoxy carbonylcyanide phenylhydrazone (FCCP) or related drugs. In a preferred embodiment, the metabolic inhibitor is oligomycin and accounts for between about 0.0001 and about 13 wt % of the total weight of the composition.

Alternatively, the metabolic inhibitor can comprise a weight percentage (wt %) of the total weight of the composition, for example between about 0.01 and about 30 wt % of the total weight of the composition. The composition may comprise a weight percentage of any amount between the about 0.01 and about 30 wt %, including, but not limited to, about 0.05 wt %, about 0.1 wt %. about 0.2 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, or about 29 wt %. Any range between the about 0.01 wt % and about 30 wt % is also contemplated, including, but not limited to between about 0.02 to about 21 wt %, between about 0.01 and about 17 wt %, between about 0.01 and about 11 wt %, and between about 0.01 and about 13 wt % of the total weight of the composition.

Still further, the metabolic inhibitor may be uniformly spread throughout the composition providing a constant release of the metabolic inhibitor as the polymer degrades or breaks down. Alternatively, the metabolic inhibitor may be non-uniformly spread throughout the composition, providing a variable release of the metabolic inhibitor as the polymer degrades. For example, the metabolic inhibitor may be present at a relatively high weight percentage near the surface of the composition and at a relatively low weight percentage within the interior of the composition. In another embodiment, the metabolic inhibitor may be present at a relatively low weight percentage near the surface of the composition and at a relatively high weight percentage within the interior of the composition.

The weight percentage of the metabolic inhibitor and/or the specific polymer used in the composition may be standard for all patients or tailored (or otherwise custom set) for a particular patient in light of one or more of the condition of the particular patient, whether the patient is immunocompromised or generates a robust immune response, the particular genetic profile of the patient (including but not limited to the specific alleles and/or genetic predisposition of the patient), and/or the particular disease, disorder, or condition of the subject requiring treatment. The particular identity and weight percentage of the metabolic inhibitor and/or particular polymer to be used can be determined by the artisan.

In some embodiments, one or more metabolic inhibitors may be incorporated within the polymer, such as within the matrix of the polymer. Alternatively, one or more metabolic inhibitors may be coated on the exterior of the polymer. In some instances, one or more metabolic inhibitors may be both incorporated within the polymer and coated onto the exterior of the polymer.

The composition may vary according to how the composition is to be used. For example, the composition may be incorporated into a synthetic tissue, such as a synthetic bone, cartilage, tendon, skin, blood, kidney, and liver. The composition may be inserted in any location within a body. Furthermore, the composition may contain an additional therapeutic agent. The additional therapeutic agent is not particularly limited and may be chemotherapies for cancer, antibiotics, small molecules, antibodies, antigens, calcium phosphate, hydroxyapatite, or bioglass.

### Methods of Use

The compositions described herein can be used in a variety of ways. In one embodiment is a method for modulating an immune response by providing the composition comprising a polymer, such as a non-bioabsorbable polymer, or a combination of polymers, and a metabolic inhibitor discussed above to a subject in need of such treatment. The non-bioabsorbable polymer may be a synthetic polymer, such as one or more or polyethylene, polyethylene copolymer, or a combination thereof. The polymer or combination of polymers may be further combined with or mixed with biologically acceptable metals and/or ceramics. The metabolic inhibitor may be a PFKFB3 inhibitor, a glycolytic inhibitor, a biguanide, a GABA-T inhibitor, an mETC inhibitor, or a combination of one or more of the metabolic inhibitors. The metabolic inhibitor may be incorporated within the matrix of the polymer and/or coated onto the surface of the polymer. In a particular embodiment, the polymer may be polyethylene or a polyethylene copolymer.

In some embodiments, the composition is provided (e.g., inserted into the body of the subject) as a synthetic tissue. Alternatively, the composition is provided (e.g., inserted into the body of the subject) as a depot. Where the composition is in the form of a synthetic tissue, the synthetic tissue may supplement or replace bone, cartilage, tendon, skin, blood, kidney, and/or liver.

The composition provided to the subject may comprise between about 0.01 wt % and about 30 wt %, or any amount or range between these values. In particular, the metabolic inhibitor may account for between about 0.02 and about 21 wt %, between about 0.01 and about 17 wt %, between about 0.01 and about 11 wt %, and between about 0.01 and about 13 wt % of the total weight of the composition.

In some embodiments, the subject may have undergone surgery, received orthopedic treatment, received ophthalmologic treatment, or may be suffering from a chronic disease. The subject may be a human or a non-human animal.

### Synthetic Tissues and Depots

The compositions described herein can be prepared in the form of a synthetic tissue, wherein the synthetic tissue comprises a polymer (such as a non-bioabsorbable polymer or a combination of polymers) and a metabolic inhibitor. The non-bioabsorbable polymer may be a synthetic polymer, such as one or more polyethylene, polyethylene copolymer, or a combination thereof. Alternatively, the polymer may further comprise or be incorporated within the synthetic tissue. The metabolic inhibitor may be a PFKFB3 inhibitor, a glycolytic inhibitor, a biguanide, a GABA-T inhibitor, an mETC inhibitor, or a combination of one or more of the metabolic inhibitors. The synthetic tissue may be in the form of bone, cartilage, tendon, skin, blood, kidney, and/or liver. The synthetic tissue may completely replace an organ, tissue, or body part of a subject or may replace part of an organ, tissue, or body part. For example, the synthetic tissue may a synthetic bone in the form of a femur and provided to a subject whose femur, or portion thereof, has been removed. In an alternative example, the synthetic tissue may be in the form of a bone and grafted onto a subject's bone to replace a portion of the subject's bone, such as a diseased, damaged, and/or injured portion of bone that has been removed.

The synthetic tissue may comprise an amount of the metabolic inhibitor as low as about 1 μM and as high as about 1 M, or any amount in between, such as, about 10 μM, about 100 μM, about 0.25 mM, about 0.5 mM, about 1 mM, about 10 mM, about 15 mM, about 20 mM, about 25 mM, about 50 mM, and about 100 mM. The synthetic tissue may comprise a range of metabolic inhibitor, such as between about 1 μM and about 1 M. Any range in between about 1 μM and about 1 M is also contemplated, including, but not limited to, between about 10 μM and about 100 mM, between about 100 μM and about 20 mM, or, in particular, between about 0.5 mM and about 15 mM.

Alternatively, the metabolic inhibitor can comprise a weight percentage (wt %) of the total weight of the synthetic tissue, for example between about 0.01 and about 30 wt % of the total weight of the synthetic tissue. The composition may comprise a weight percentage of any amount between the about 0.01 and about 30 wt %, including, but not limited to, about 0.05 wt %, about 0.1 wt %, about 0.2 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, or about 29 wt %. Any range between the about 0.01 wt % and about 30 wt % is also contemplated, including, but not limited to between about 0.02 to about 21 wt %, between about 0.01 and about 17 wt %, between about 0.01 and about 11 wt %, and between about 0.01 and about 13 wt % of the total weight of the composition.

The synthetic tissue may comprise a PFKFB3 inhibitor as the metabolic inhibitor. The PFKFB3 inhibitor may be one or more of 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15). In a preferred embodiment, the metabolic inhibitor is 3PO and accounts for between about 0.02 and about 21 wt % of the total weight of the synthetic tissue.

The synthetic tissue may comprise a glycolytic inhibitor as the metabolic inhibitor. The glycolytic inhibitor may be one or more of 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, or NHI1. In a preferred embodiment, the metabolic inhibitor is 2DG and accounts for between about 0.01 and about 17 wt % of the total weight of the synthetic tissue.

The synthetic tissue may comprise a biguanide as the metabolic inhibitor. The biguanide may be one or more of metformin, buformin, or phenoformin. In a preferred embodiment, the metabolic inhibitor is metformin and accounts for between about 0.01 and about 11 wt % of the total weight of the synthetic tissue.

The synthetic tissue may comprise a GABA-T inhibitor as the metabolic inhibitor. The GABA-T inhibitor may be one or more of aminooxyacetic acid, vigabatrin, gabaculine, phenelzine, phenylethylidinchydrazine (PEH), rosmarinic acid, valproic acid, ethanolamine-O-sulfate (EOS), and cycloserine. In a preferred embodiment, the metabolic inhibitor is metformin and accounts for between about 0.01 and about 13 wt % of the total weight of the synthetic tissue.

The synthetic tissue may comprise an mETC inhibitor as the metabolic inhibitor. The mETC inhibitor may be a rotenoid or a macrolide. The rotenoid may be one or more of rotenone, rotenol, deguelin, dehydrodegulein, tephrosin, or sumatrol. The macrolide may be one or more of oligomycin, azithromycin, clarithromycin, or erythromycin. Alternatively, the mETC inhibitor may be FCCP. In a preferred embodiment, the metabolic inhibitor is oligomycin and accounts for between about 0.0001 and about 13 wt % of the total weight of the synthetic tissue.

The metabolic inhibitor may be incorporated within the matrix of the polymer of the synthetic tissue. Alternatively, the metabolic inhibitor may be coated onto the surface of the polymer of the synthetic tissue. In another embodiment, the metabolic inhibitor may be both incorporated into the bioabsorbable matrix and coated onto the surface of the polymer of the synthetic tissue. In such instances, the metabolic inhibitor incorporated within the matrix of the polymer may be the same or different than the metabolic inhibitor coated onto the surface of the polymer. The particular species of metabolic inhibitor incorporated into the polymer matrix or coated onto the surface of the polymer can be determined to accommodate the particular requirements of a treatment protocol. An additional therapeutic agent may also be delivered if present in the synthetic tissue.

The synthetic tissue may be produced through three-dimensional printing technology. Following incorporation of the metabolic inhibitor in the polymer by melt-blending, filaments may be extruded. Computer-aided design, which may be patient-derived, utilize extruded filaments in a multi-dimensional printer to make synthetic tissues. Multiple dimensional printers may be 3D or 4D printers. Alternatively, printed polymeric implants may have their surfaces coated by methods not limited to physical or chemical binding.

Alternatively, the compositions described herein can be prepared in the form of a depot. which does not possess the shape, structure, or function of an organ, tissue, or body part of the subject. The size of the depot is not particularly limited and may range in diameter from a nanometer scale to a centimeter scale In this manner, the depot delivers one or more metabolic inhibitor to the subject as the polymer decomposes or the inhibitor is pushed out via osmotic pressure. An additional therapeutic agent may also be delivered if present in the depot. The depot can be inserted into the body cavity of a subject, such as under the skin or within a body cavity. The particular polymers and metabolic inhibitors for the depot are similar to those for the synthetic tissue described above.

### EXAMPLES

The following example is provided to further illustrate the fusion peptide disclosed herein but should not be construed as in any way limiting its scope.

### Example 1

### Metabolic Remodeling by Polyethylene (PE) Particles

This example demonstrates the molecular mechanism underlying metabolic reprogramming in inflammation and fibrosis following exposure to degraded non-absorbable polyethylene particles. Ultra-high molecular weight polyethylene particles were obtained and determined to be endotoxin-free. Concentrations of 100 ng/mL lipopolysaccharide (LPS) from E. coli O111:B4 (Millipore Sigma) and 1.25 mg/mL ultra-high molecular weight polyethylene particles were used. 3DO, 2DG, and a.a. were used for glycolytic inhibition.

Bioluminescence was measured using an IVIS Spectrum in vivo imaging system (PerkinElmer) after addition of 150 μg/mL of D-luciferin (PerkinElmer). Living Image (Version 4.5.2, PerkinElmer) was used for acquiring bioluminescence on the IVIS Spectrum. Standard ATP/ADP kits (Sigma-Aldrich) containing D-luciferin, luciferase, and cell lysis buffer were used according to manufacturer's instructions. Luminescence at integration time of 1,000 ms was obtained using the SpectraMax M3 Spectrophotometer (Molecular Devices) and SoftMax Pro software (ver. 7.0.2).

BMDMs derived from C57BL/6J mice (Jackson Laboratories) of 3-4 months were used. Mouse embryonic fibroblasts (NIH 3T3 cells, hereafter “MEFs”) were stably transfected with a Sleeping Beauty transposon plasmid (pLuBIG) having a bidirectional promoter driving an improved firefly luciferase gene (fLuc) and a fusion gene encoding a Blasticidin-resistance marker (BsdR) linked to eGFP (BGL). For temporal (IVIS) experiments lasting 12 days, 5,000 BGL cells were initially seeded in each well of a 96-well tissue culture plate in 200 μL if complete medium. For ATP, crystal violate, and Seahorse assays, 20,000 wild-type MEFs were seeded. For ATP, crystal violate, and cytokine/chemokine assays, 50,000 BMDMs were seeded. 60,000 BMDMs were seeded for the Seahorse assays. For IVIS experiments with glycolytic inhibitors, 20,000 BGL cells were initially seeded. Complete medium comprised DMEM medium, 10% heat inactivated fetal bovine serum (FBS), and 100 U/mL penicillin/streptomycin (all from ThermoFisher Scientific).

Cell viability was measured using the crystal violet staining assay at room temperature. 50 μL media containing cells is incubated with 150 μL of 99.9% methanol for 15 minutes. 100 μL of 0.5% crystal violet (25% methanol) is added for 20 minutes. Each well is emptied and washed twice with 200 μL phosphate buffered saline for 2 minutes. Absorbance (optical density) was acquired at 570 nm using the SpectraMax M3 Spectrophotometer (Molecular Devices) and SoftMax Pro software (ver. 7.0.2).

To determine the metabolic pathways responsible for the observed bioenergetic changes, Seahorse assays were used to measure oxygen consumption rate (OCR), extracellular acidification rate (ECAR) and lactate-linked proton efflux rate (PER) in a customized medium (pH 7.4). Basal measurements of OCR, ECAR, and PER were obtained in real time using the Seahorse XFe-96 Extracellular Flux Analyzer (Agilent Technologies). Prior to running the assay, cell culture medium was washed with and replaced by the Seahorse XF DMEM medium (pH 7.4) supplemented with 25 mM D-glucose and 4 mM glutamine. The Seahorse plates were equilibrated in a non-CO₂ incubator for an hour prior to the assay. The Seahorse ATP rate and cell energy phenotype assays were run according to manufacturer's instruction and all reagents for the Seahorse assays were sourced from Agilent Technologies. Wave software (Version 2.6.1) was used to export Seahorse data directly as means±standard deviation (SD).

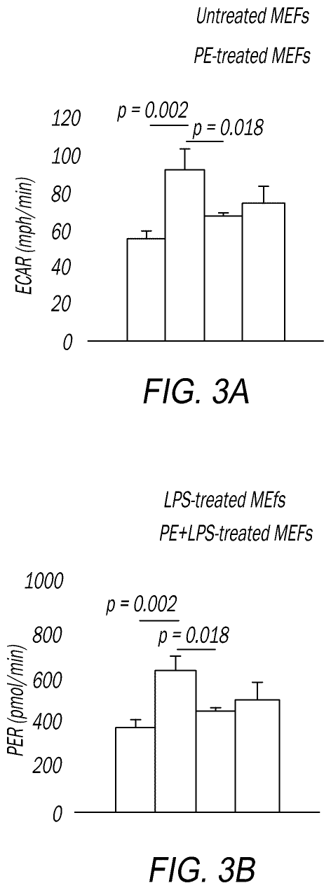

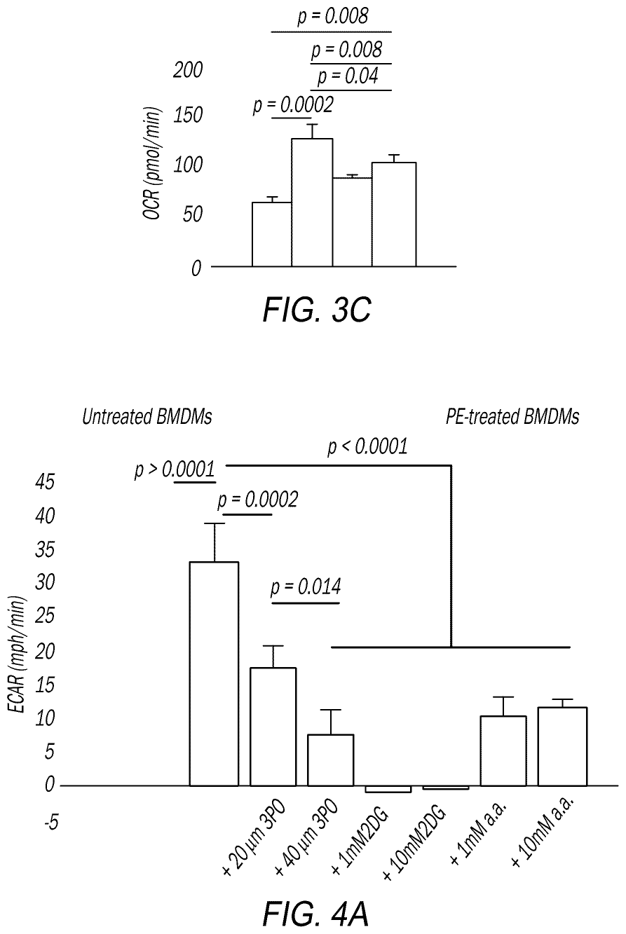

Seahorse assays measures ECAR as an index of glycolytic flux, OCR as an index of oxidative phosphorylation and PER as an index of monocarboxylate transporter function in live cells, and are used to assess for metabolic reprogramming. Following exposure to LPS alone, MEFs did not show any change in ECAR, PER, or OCR as compared to untreated controls. However, exposure to PE resulted in 1.7-, 1.7-, and 2-fold increases in ECAR, PER, and OCR, respectively, as compared to untreated MEFs. Exposure to LPS alone showed no effect on ECAR, PER, or OCR. Exposure to PE+LPS showed no effect on ECAR or PER but did increase OCR by 1.6-fold as compared to the untreated MEFs. (FIGS. 3a-c)

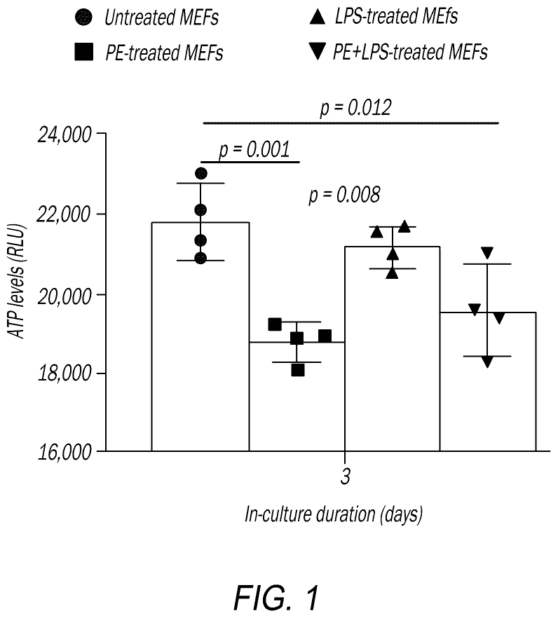

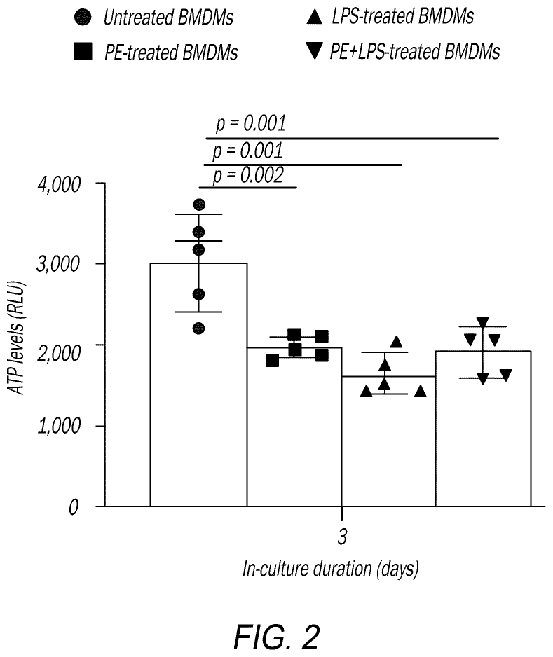

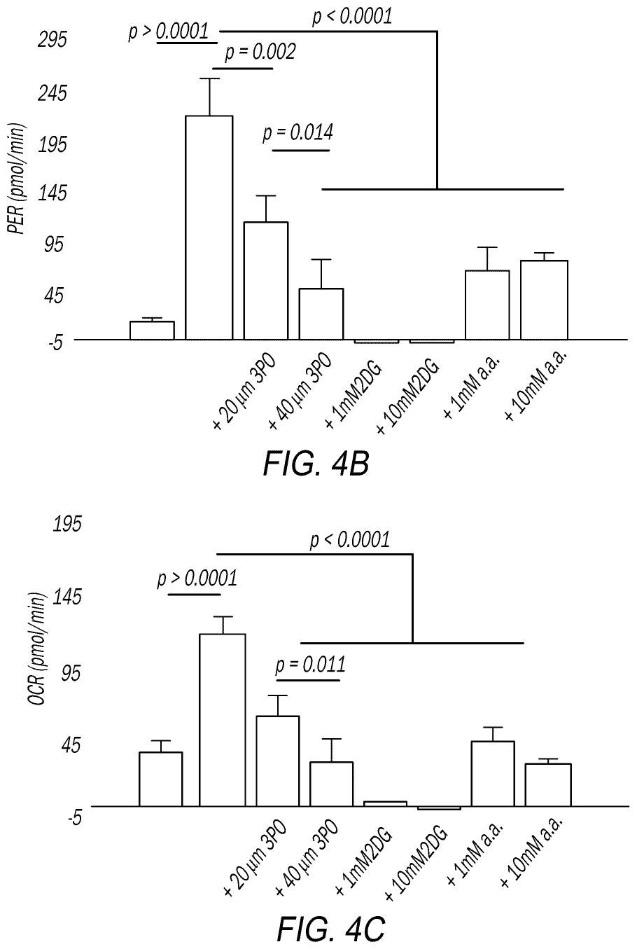

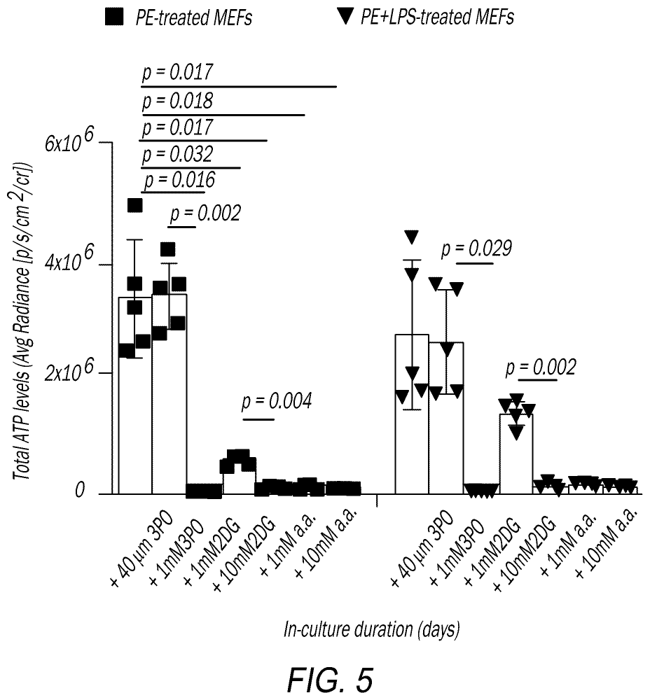

To determine the effect of polyethylene particles (PE) on bioenergetic levels of cells, MEFs and BMDMs were cultured in the absence and presence of PE and LPS. PE consistently lowered ATP levels in PE-treated MEFs, but LPS had no effect up to 12 days of exposure. (Data not shown.) However, in lysed cells, which is more sensitive than assays in whole cells, total ATP levels following exposure to PE was 1.2-fold lower than untreated MEFs, as compared to a 1.1-fold lower ATP level seen in MEFs exposed to PE and LPS. LPS alone did not lower ATP levels. (FIG. 1) In contrast, BMDMs treated with PE, LPS, or PE+LPS showed reduced bioenergetic levels of 1.5-, 1.8-, and 1.6-fold as compared to untreated cells on Day 3. (FIG. 4) Exposure to PE and/or LPS did not impact cell viability in a statistically significant manner for either the MEFs or BMDMs. (Data not shown.) Similarly, treatment of the MEFs or BMDMs with the metabolic inhibitors 3DO, 2DG, and a.a., did not lower cell viability. (Data not shown.) However, the metabolic inhibitors did decrease bioenergetic levels (as measured by total ATP levels) in both PE and PE+LPS treated MEFs in a dose dependent manner. (FIG. 5)

Cytokine and chemokine levels were measured using a MILLIPLEX MAP mouse magnetic bead multiplex kit (MilliporeSigma) to assess for IL-6, MCP-1, TNF-α, IL-1β, IL-4, IL-10, IFN-γ, and 1L-13 protein expression in supernatants. Data was acquired using Luminex 200 (Luminex Corporation) by the xPONENT software (Version 3.1, Luminex Corporation). Using the glycolytic inhibitor, 3PO, expectedly decreased cytokine values to <3.2 pg/mL in some experiments. For statistical analyses, those values were expressed as 3.1 pg/mL. Values exceeding the dynamic range of the assay, in accordance with manufacturer's instruction, were excluded. Additionally, IL-6 ELISA kits (RayBiotech) for supernatants were used according to manufacturer's instructions.

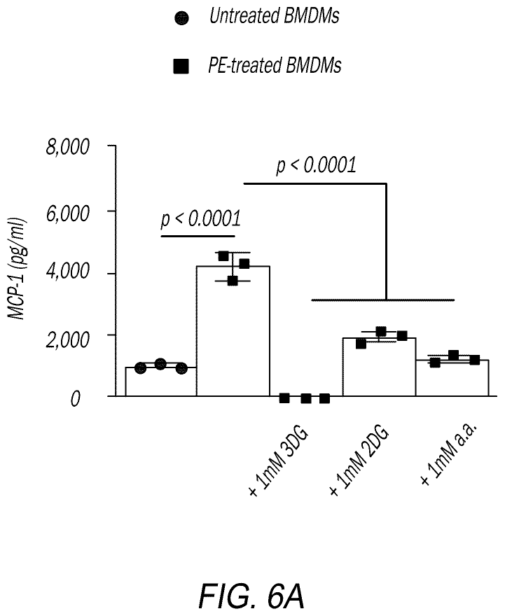

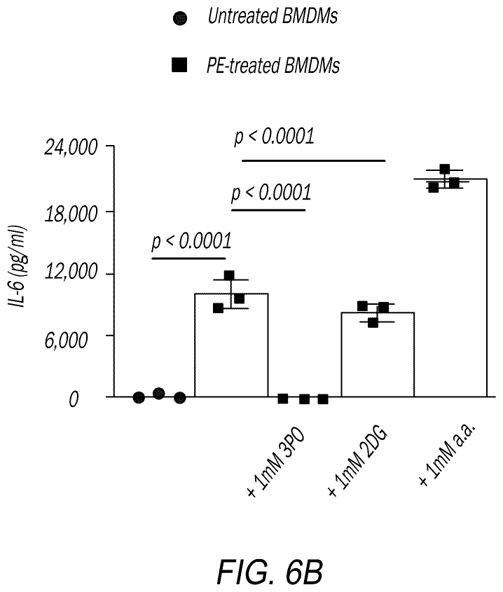

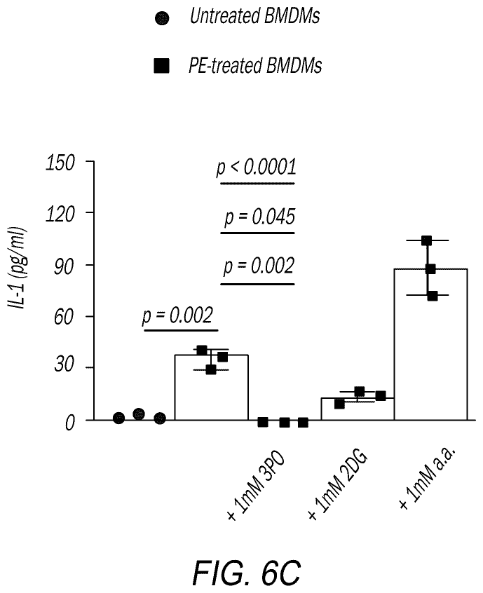

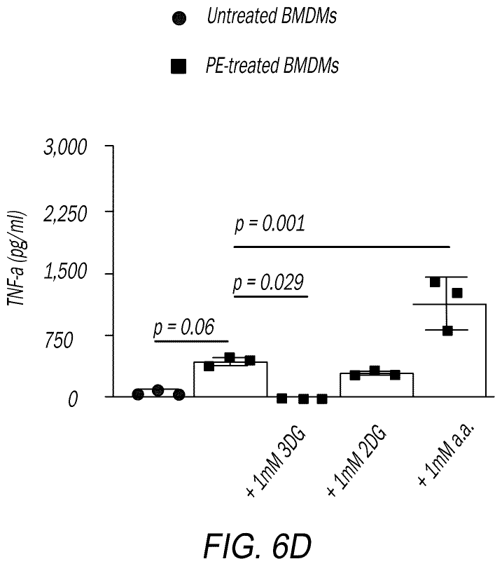

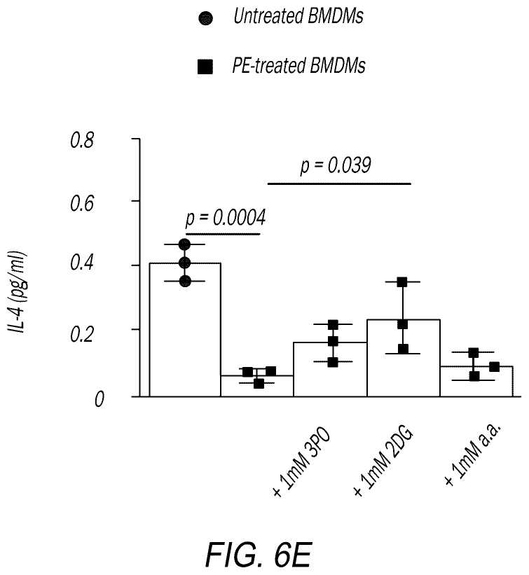

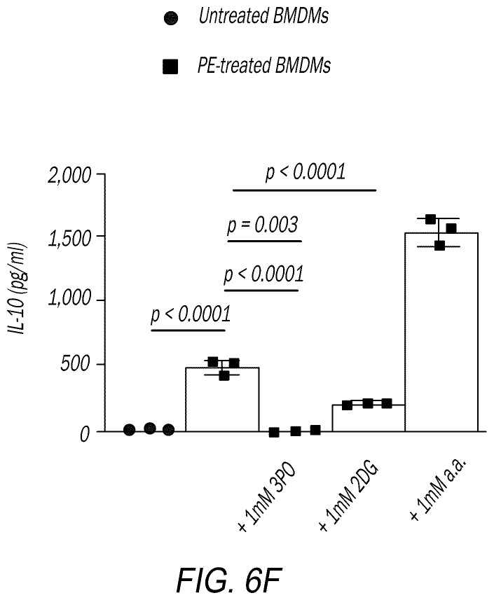

Addition of 1 mM metabolic inhibitors (c.g., 3PO, 2DG, and aminooxyacetic acid) decreased expression of pro-inflammatory cytokines MCP-1, IL-6, IL-1β, and TNF-α in BMDMs exposed to ultra-high molecular weight PE particles (FIGS. 6a-d, respectively). The same metabolic inhibitors increased IL-4 in BMDMs exposed to ultra-high molecular weight PE particles (FIG. 6e). Only aminooxyacetic acid increased IL-10 levels in BMDMs exposed to ultra-high molecular weight PE particles (FIG. 6f). These data demonstrate that addition of metabolic inhibitors reduces inflammation and expression of proinflammatory cytokines through metabolic remodeling.

### Example 2

### Oxidative Phosphorylation is Required for Immune Cell Activation

This example demonstrates the importance of oxidative phosphorylation underlying immune cell activation by polyethylene wear particles. Preparation of ultra-high molecular weight polyethylene particles are described in Example 1 above. The bioenergetics measurements, Seahorse assay, crystal violet assay, and cytokine and chemokine assays were conducted as described in Example 1. Inhibitors rotenone, metformin, oligomycin, and antimycin A were obtained from MilliporeSigma. The reagents were reconstituted in complete medium (described above).

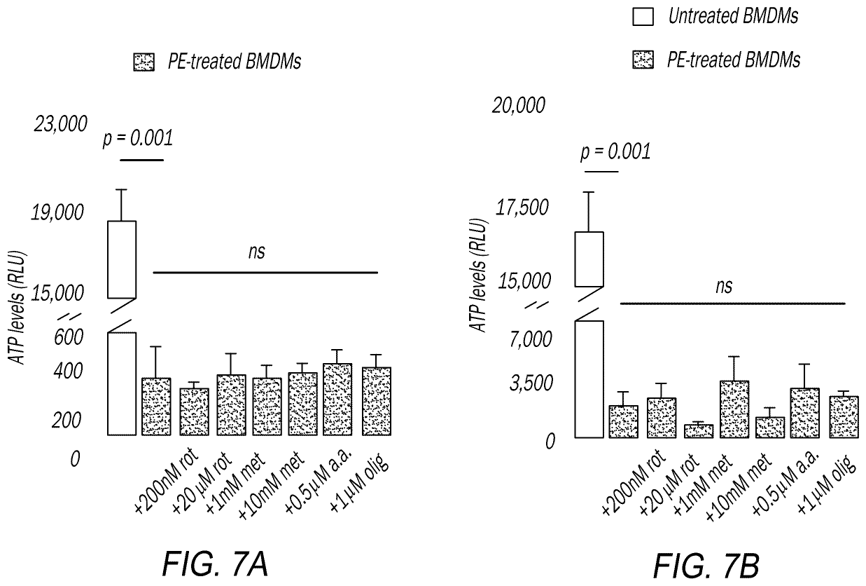

The bioenergetic (ATP) levels of primary BMDMs and MEFs treated with PE in the absence or presence of inhibitors of mitochondrial respiration were measured. BMDMs and MEFs unexposed to PE show high basal ATP levels, which are significantly reduced upon exposure to PE. Mitochondrial respiration inhibitors did not block or reverse the decreased ATP levels. (FIG. 7a: BMDMs; FIG. 7b: MEFs) Specific inhibition of complex I by rotenone or metformin, or inhibition of complex III or V by a.a. or oligomycin, respectively, did not impact ATP levels in PE-exposed cells.

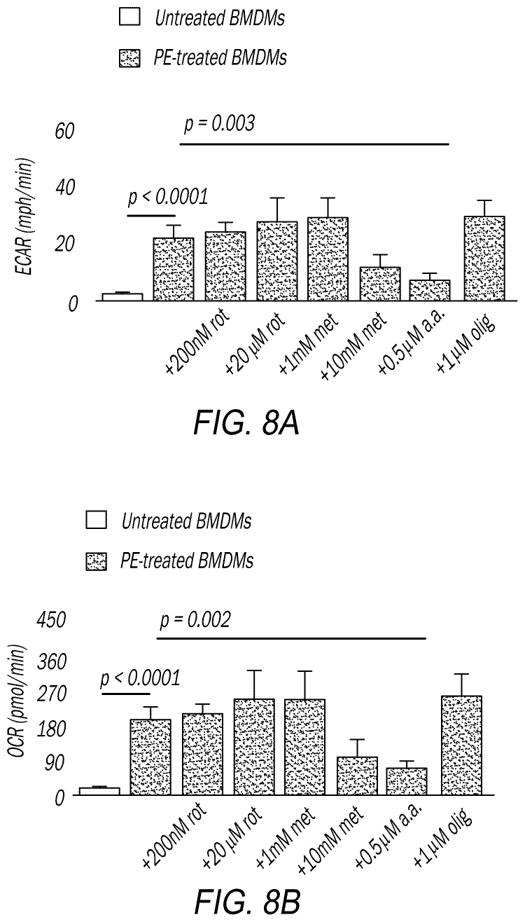

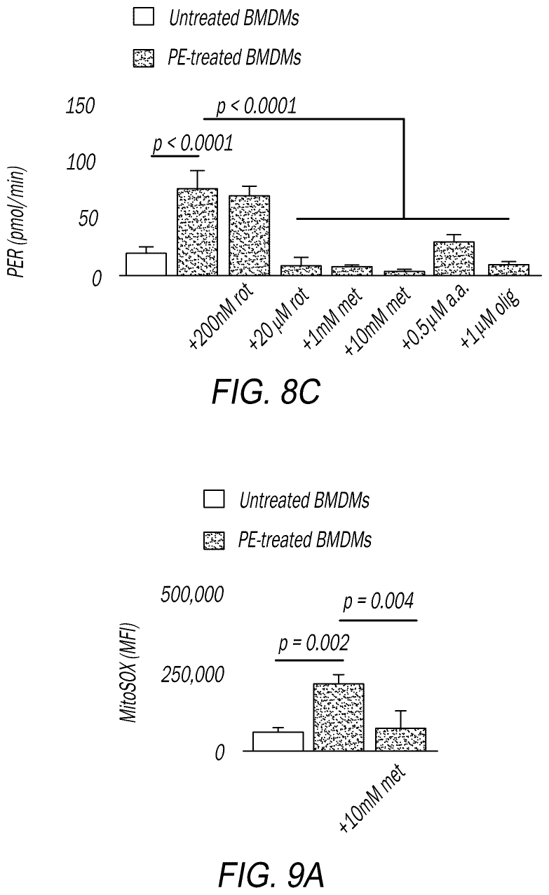

Primary macrophages exposed to PE showed an increase ECAR, PER, and OCR. The presence of rotenone did not impact ECAR or PER levels but did decrease OCR in a dose-dependent manner. Metformin and a.a. reduced ECAR, PER, and OCR in PE-exposed BMDMs, but oligomycin only affected OCR levels. (FIGS. 8a-c) Inhibition of mitochondrial respiration did not reduce cell viability. (Data not shown.)

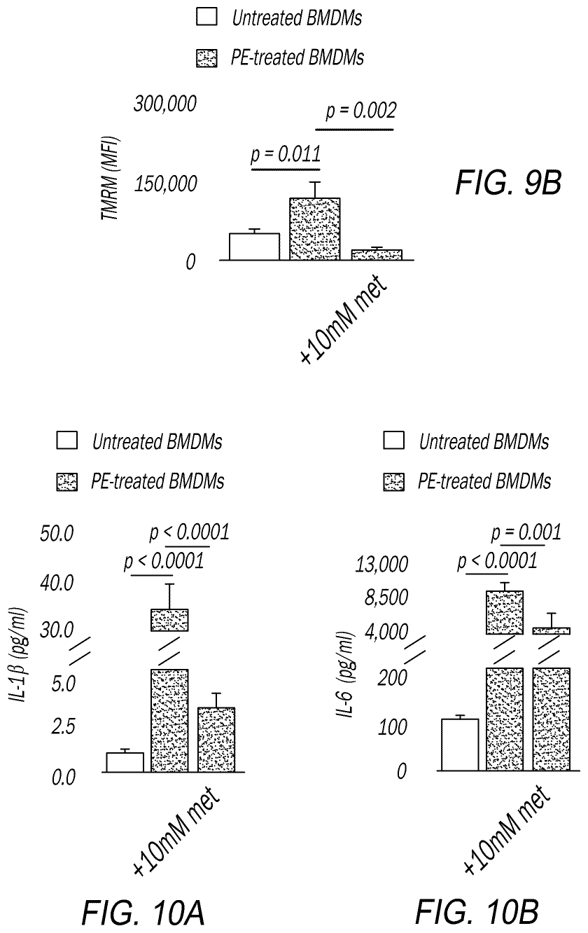

To test whether increased OCR at complex I (FIG. 8c) fueled ROS production in the mitochondrion, macrophages were stained with mitoSOX Red. Exposure to LPS or PE particles increased mitochondrial reactive oxygen species (ROS) relative to untreated macrophages. Addition of metformin decreased mitochondrial ROS compared to macrophages exposed to only PE. Mitochondrial membrane potential is critical for reverse electron transport (RET) at complex I, and subsequent generation of mitochondrial ROS. Tetramethylrhodamine methyl ester (TMRM staining demonstrates that LPS and PE particles increase mitochondrial membrane potential relative to untreated cells, and metformin decreases the elevated mitochondrial membrane potential. (FIG. 9)

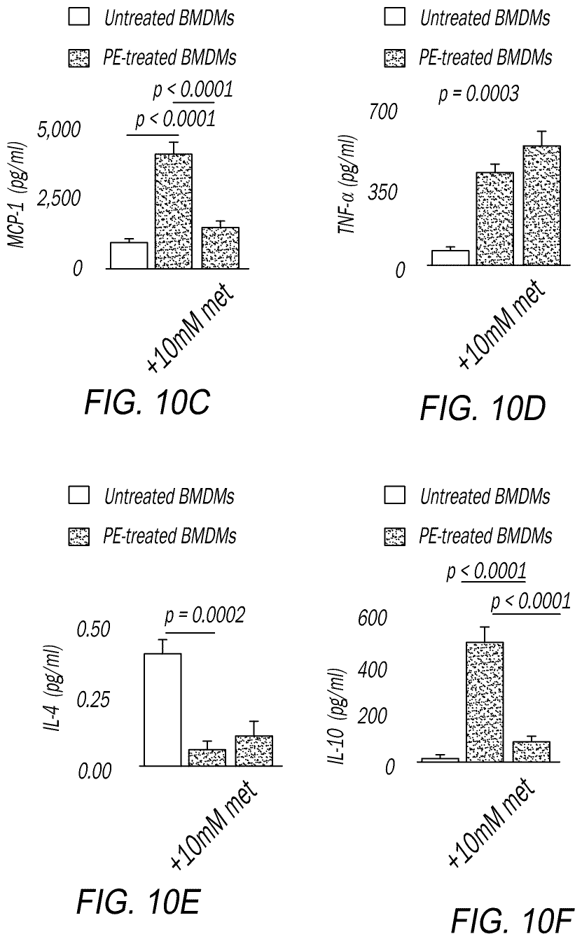

As shown in Example 1 above, exposure to PE increases proinflammatory cytokines in comparison to untreated cells. Metformin decreases elevated IL-1β, IL-6, and MCP-1. (FIGS. 10a-c) However, TNF-α expression is not mitigated by metformin. (FIG. 10d) Metformin did not increase anti-inflammatory cytokines IL-4 or IL-10 (FIGS. 10e-f).

The use of the terms “a” and “an” and “the” and “at least one” and similar referents in the context of describing the fusion peptide and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the fusion peptide are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the fusion peptide to be practiced otherwise than as specifically described herein. Accordingly, the fusion peptide described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

What is claimed is:

1. A composition comprising a polymer and a metabolic inhibitor,
wherein the polymer is selected from the group consisting of polyethylene, polyethylene copolymer, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a mitochondrial electron transport chain inhibitor (mETC), a biguanide, a γ-aminobutyrate aminotransferase (GABA-T) inhibitor, and combinations thereof,
wherein the composition comprises between about 1 μM and about 1 M of the metabolic inhibitor and
wherein the inhibitor comprises between about 0.01 wt % to about 30 wt % of the total weight of the composition.

2. (canceled)

3. The composition according to claim 1, wherein the composition comprises between about 0.5 mM and about 15 mM of the metabolic inhibitor.

4. (canceled)

5. The composition according to claim 1, wherein the metabolic inhibitor comprises between about 0.02 wt % to about 21 wt %, between about 0.01 wt % and about 17 wt %, between about 0.01 wt % and about 11 wt %, and between about 0.01 wt % and about 13 wt % of the total weight of the composition.

6. The composition according to claim 1, wherein the metabolic inhibitor is a PFKFB3 inhibitor.

7. The composition according to claim 6, wherein the PFKFB3 inhibitor is selected from the group consisting of 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidinecarboxamide (AZ76), (2S)-N-[4-[[3-cyano-1-(2-methylpropyl)-1H-indol-5-yl]oxy]phenyl]-2-pyrrolidine carboxamide (AZ 26), and 1-(4-pyridinyl)-3-(2-quinolinyl)-2-propen-1-one (PFK15).

8. The composition according to claim 7, wherein the PFKFB3 inhibitor is 3PO and comprises between about 0.02 and 21 wt % based on the total weight of the composition.

9. The composition according to claim 1, wherein the metabolic inhibitor is a glycolytic inhibitor.

10. The composition according to claim 9, wherein the glycolytic inhibitor is selected from the group consisting of 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, and NHI1.

11. The composition according to claim 11, wherein the glycolytic inhibitor is 2DG and comprises between about 0.01 and about 17 wt % based on the total weight of the composition.

12. The composition according claim 1, wherein the metabolic inhibitor is a biguanide.

13. The composition according to claim 12, wherein the biguanide is selected from the group consisting of metformin, buformin, and phenoformin.

14. The composition according to claim 13, wherein the biguanide is metformin and comprises between about 0.01 and about 11 wt % based on the total weight of the composition.

15. The composition according to claim 1, wherein the metabolic inhibitor is a GABA-T inhibitor.

16. The composition according to claim 15, wherein the GABA-T inhibitor is selected from the group consisting of aminooxyacetic acid, vigabatrin, gabaculine, phenelzine, phenylethylidinehydrazine (PEH), rosmarinic acid, valproic acid, ethanolamine-O-sulfate (EOS), and cycloserine.

17. The composition according to claim 16, wherein the GABA-T inhibitor is aminooxyacetic acid and comprises between about 0.01 and about 13 wt % based on the total weight of the composition.

18. The composition according to claim 1, wherein the metabolic inhibitor is an mETC inhibitor.

19. The composition according to claim 18, wherein the mETC inhibitor is selected from the group consisting of rotenone, rotenol, deguelin, dehydrodegulein, tephrosin, sumatrol, oligomycin, azithromycin, clarithromycin, erythromycin, and trifluoromethoxy carbonylcyanide phenylhydrazone.

20. The composition according to claim 19, wherein the mETC inhibitor is oligomycin and comprises between about 0.0001 and about 13 wt % based on the total weight of the composition.

21. The composition according to claim 1, wherein the metabolic inhibitor is incorporated into the polymer.

22. The composition according to claim 1, wherein the metabolic inhibitor is coated onto the polymer.

23. (canceled)

24. (canceled)

25. The composition according to claim 1, further comprising an additional therapeutic agent selected from the group consisting of chemotherapies for cancer, antibiotics, small molecules, antibodies, antigens, calcium phosphate, hydroxyapatite, and bioglass.

26. (canceled)

27. A method for modulating an immune response comprising providing to a subject in need thereof a composition comprising a polymer and a metabolic inhibitor,
wherein the polymer is selected from the group consisting of polyethylene, polyethylene copolymer, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, a mitochondrial electron transport chain (mETC) inhibitor, and combinations thereof,
wherein the composition comprises between about 1 μM and about 1 M of the metabolic inhibitor and
wherein the inhibitor comprises between about 0.01 wt % to about 30 wt % of the total weight of the composition.

28. (canceled)

29. The method according to claim 27, wherein the composition is a synthetic tissue or a depot.

30. The method according to claim 29, wherein the synthetic tissue is selected from the group consisting of bone, cartilage, tendon, skin, blood, kidney, and liver.

31. (canceled)

32. The method according to claim 27, wherein the subject is selected from the group consisting of a surgery subject, an orthopedic subject, an ophthalmologic subject, a subject suffering from a chronic disease, and injury.

33. (canceled)

34. (canceled)

35. A synthetic tissue comprising a polymer and a metabolic inhibitor,
wherein the polymer is selected from the group consisting of polyethylene, polyethylene copolymer, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, a mitochondrial electron transport chain (mETC inhibitor), and combinations thereof,
wherein the composition comprises between about 1 μM and about 1 M of the metabolic inhibitor and
wherein the inhibitor comprises between about 0.01 wt % to about 30 wt % of the total weight of the composition.

36. The synthetic tissue according to claim 35, wherein the synthetic tissue is selected from the group consisting of bone, cartilage, tendon, skin, blood, kidney, and liver.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)
